(19) United States
(12) Patent Application Publication
(10) Pub. No.: US 2011/0054524 A1
(43) Pub. Date: Mar. 3, 2011

(54) IMPLANTABLE PROSTHETIC CORD

(75) Inventors: David Beevers, Yorkshire (GB); Atif Mostafa Abdel-Kader Nada, South Wales (GB); Lauren Calloway, Yorkshire (GB)

(73) Assignee: XIROS LIMITED, Yorkshire (GB)

(21) Appl. No.: 12/921,089

(22) PCT Filed: Mar. 4, 2009
(86) PCT No.: PCT/GB09/50214

§ 371 Date: Nov. 19, 2010

(30) Foreign Application Priority Data
Mar. 4, 2008 (GB) 0804018.0

(51) Int. Cl. A61B 17/04

(52) U.S. Cl. 606/228

(57) ABSTRACT

A medical cord and surgical procedure for the connection of a first tissue site to a second tissue site. The cord comprises a main length having a width and end portions having respective widths being less than the width of the main length. This provides a medical cord that is easy to manipulate during surgical procedures in which the cord may be required to be threaded through apertures and tunnels. The cord is particularly suitable to attach soft tissue, including ligaments and tendons to bone anchorsites and provides a prosthetic scaffold to facilitate tissue ingrowth at the repair site.

The present invention relates to a surgical cord configured to secure a first biological tissue site to a second biological tissue site and a method of surgical repair.

Soft tissue (tendon and ligament) ruptures are common amongst sports professionals and the elderly for example. A number of different procedures have been developed to repair soft tissue tears being specific to the damaged, anatomical region.

In particular, a number of different connecting devices have been proposed to reconnect the torn tissue to its original anchor point which may typically comprise a bone site. Important considerations for these connecting devices such as sutures and surgical tapes include strength and ease of manipulation by the surgeon during repair surgery. Surgical tapes in particular comprise bioabsorbable or non-bioabsorbable materials from natural or artificial sources including for example gut, silk, cotton, polyester and specific ultra high molecular weight polyethylene (UHMWPE). A further important characteristic of the cord is its resistance to ‘tissue pull-through’ when under load. Tissue pull-through refers to the process of the cord or tape pulling or cutting through the tissue under tensile load when the joint is manipulated post surgery. Cords having small diameters or widths and sharp edges are disadvantageous as they are more susceptible to tissue pull-through. Cords having a greater width are however often difficult to manipulate by a surgeon who is typically required to thread the ends of the cord through narrow apertures and form knotted attachments.

US-2005/0192631 discloses a suture tape construct comprising a braided high strength surgical suture material. A tubular braided suture extends along the length of the tape to provide a backbone to the construct. The tape comprises an additional flat braided middle portion through which the tubular braided suture is threaded. Transition sections at each end of the flat braid are tapered to allow the suture tape to pass through apertures during surgical procedures and provide a means of anchoring the central tubular suture to the flat tape. The construct comprises UHMWPE fibre blended with one or more long chain synthetic polymers such as polyester to provide the desired strength.

US 2004/0078089 discloses a textile prostheses for use as a surgical implant in which a main portion comprises at least one anchorage body portion designed to accommodate tensile loadings and being resistant to stretch when placed under load. The anchoring portions may comprise tapers or a width being less than the main body.

There is therefore a need for surgical cord or tape that is both easy to manipulate by a surgeon during surgical procedures whilst exhibiting the required strength and tissue pull through resistance.

The present invention provides a surgical cord prostheses exhibiting strength characteristics suitable for use as a means of connection between two biological tissue sites. In particular, the present surgical cord is ideally suited to reconnect soft tissue in the form of tendons, ligaments and the like, to a bone site. The tape comprises a wide flat main body that exhibits tissue pull through resistance and has adjacent thin end regions to allow the cord to be manipulated during surgical procedures.

According to a first aspect of the present invention there is provided a surgical cord comprising: a plurality of interwoven yarn strands comprising warps and wefts extending over a woven main length of the cord, the main length having a width; two end regions formed by said yarn strands, a width of said end regions being less than the width of the main length wherein said warps extend from said main length in to each end region; and wherein the main length of the cord comprises a flat, substantially planar profile.

Preferably, the yarn strands comprise twisted polyester fibres such that the surgical cord may comprise exclusively polyester yarn strands. Due to the cord construction, the requirement for UHMWPE which is commonly incorporated within surgical tape to provide tensile strength, is avoided. The present cord is therefore easy to manufacture, comprising a single material.

The main length of the cord is woven however the end regions may be interlaced, woven, knitted or braided. The yarn strands of the main length are interwoven and comprise warps (aligned with the longitudinal axis of the cord) and wefts (aligned transverse to the longitudinal axis of the cord), both warps and wefts extending along the cord main length. According to specific implementations, the cord comprises a plurality of groups of three wefts interwoven with perpendicular aligned groups of three wefts to form a woven mesh-like structure. This open weave structure provides a scaffold promoting tissue ingrowth and post surgical repair.

The warps, and optionally the wefts, extend from the main length into each end region. The end regions may be braided and in particular the warps in the end region, extending from the main length, may be braided.

The number of warps in the end regions may be equal to the number of warps in the main length or the number of warps may decrease along the end regions in a direction away from the main length. Preferably, the spacing of the yarn strands in each end region is less than the spacing between the yarn strands in the main length so as to provide a more dense cord at the main length.

The wefts may extend from the main length into each end region. The spacing between wefts within each end region may be greater or equal to the spacing of the wefts in the main length. Optionally, the wefts may terminate at the transition between the main length and end regions. Optionally, the wefts extending into each end region may be braided.

Optionally, the warps are bound within the end regions by twisting or plying. Alternatively, the warps extending from the woven tape section into the end regions may be bound by knitting or braiding. Alternatively the warps may be bound together by additional embroidery. Alternatively the warps may be bound together by wrapping, the warps acting as a core. The wrapping may be provided by the warps extending from the woven tape main body or additional yarn may be used. The features in the end regions in each of the embodiments described above may be formed from the wefts alone, or the warps alone, or a combination of the wefts plus the warps or by using an additional material. In each case all or a proportion of the wefts or warps may be used.

Alternatively, the main body of the cord may incorporate twisted, plyed, braided or plaited yarns as warps which extend from the woven main body section into the end region. The yarn in the end regions may be bound together by further twisting or plying or by additional braiding, plaiting, knit braiding, linking, knotting and/or wrapping. Other wraps running parallel can be incorporated into this twist, ply and/or braid etc. or removed at the end of the main body section.

Optionally, additional braided, plaited, twisted, plyed, or woven yarns may be embroidered or sewn onto the either or both of the upper or lower face of the flat substantially planar cord. These additional yarns may then extend beyond the main body into the end regions and run substantially parallel with the warps of the main body. Optionally, warps extending from the main body into the end sections may be removed to reduce the bulk of material at these end sections.

Alternatively, braided, plaited, twisted, plyed or woven yarns may be threaded amongst the woven warps and wefts extending within the main body. These additional yarns may extend into the end regions. Some or all of the warps extending within the main body may not extend into each end region.

The cord main length is flat having a substantially wide planar tape like structure. This configuration resists cutting through the soft tissue under load. The end regions may also be flat and comprise a substantially uniform width and thickness over the respective length of each end region. Reference within this specification to a ‘substantially planar profile’ includes a cord that is many orders of magnitude wider that it is thick. For example, the cord, at the main length, may be formed from a single layer of interwoven strands or a plurality of layers that are overlaid or flattened again one another so as to provide a tape like profile. In particular the main length may be formed from a tubular woven textile that has been flattened with the walls of the tube in contact such that there is no internal hollow cavity between the otherwise tubular cord. Where the cord is multilayered (initially tubular), it is configured to maintain its resulting planar profile via initial processing such as pressing or ironing flat optionally involving heat treatment. Optionally, the layers may be attached together via embroidery, stitching and the like

According to a second aspect of the present invention there is provided a method of surgical repair comprising: securing a cord to a first biological tissue site, said cord comprising: a plurality of interwoven yarn strands comprising warps and wefts extending over a woven main length of the cord, the main length having a width; two end regions formed by said yarn strands, a width of said end regions being less than the width of the main length wherein said warps extend from said main length in to each end region; and wherein the main length of the cord comprises a flat, substantially planar profile; securing said cord at a second biological tissue site; wherein said cord extends between said first and second tissue sites.

The present method is advantageous for the reconstruction and attachment of soft tissue to a bone site as part of a surgical repair procedure.

The method preferably comprising forming at least one hole at the first tissue site and threading the cord through the hole to form a loop. The method may further comprise drilling at least one hole or through-bore at the bone site and threading the cord through the bone tunnel. The ends of the cord may then be secured in position at the bone site by knotting the ends of the cord together to prevent retraction as the joint is manipulated and the cord placed under tensile load.

According to a third aspect of the present invention there is provided a method of connecting a first biological tissue site to a second biological tissue site comprising: securing a cord to a first tissue site by threading said cord through at least one hole formed in said first tissue site, said cord comprising a plurality of interwoven yarn strands comprising warps and wefts extending over a woven main length of the cord, the main length having a width; two end regions formed by said yarn strands, a width of said end regions being less than the width of the main length wherein said warps extend from said main length in to each end region; and wherein the main length of the cord comprises a flat, substantially planar profile; securing said cord to a second tissue site; wherein said cord extends between said first and second tissue sites.

The present invention is particularly suitable to reconstruct a torn rotator cuff by reconnecting the rotator cuff with the humeral head via the present surgical tape.

Preferably, the method comprises forming at least two holes in the cuff tissue and looping the cord through the holes such that the cord extends from the tissue as two strands positioned side by side. This would involve threading the cord through the cuff tissue to form an n-shaped loop. This has the advantage of forming a strong couple between the prosthetic and the biological tissue and obviates the use of additional securing sutures according to prior art methods of attachment. The method further comprises securing the cord to the humeral head by first drilling holes through a region of the humeral head, threading the end regions of cord through the bone tunnels, and knotting the cord ends together to provide fixation.

The width of cord along the main length is configured such that when arranged in this side-by-side configuration the thickness of the prosthetic rotator cuff is approximately equal to the width of the cuff tissue to which it is attached.

Both ends of the cord are tapered and sealed allowing the cord to be easily manipulated and pulled through the soft tissue and bone tunnels. The cord ends provide ease of knotting and the reduced bulk of material at the ends allows for the creation of smaller knots. The thickness of the cord, particularly when arranging in the side-by-side configuration and looped through the tissue via two holes, acts as an extension to the torn rotated cuff and provides excellent intrinsic strength and allows early mobilisation. The combined dual cord width, in the side-by-side arrangement, also serves to inhibit joint dislocation by effectively restricting the joint/bone movement due to the increased cord-bone contact surface area.

Other features and advantages of the present invention will become apparent from the following description with reference to the accompanying drawings in which:

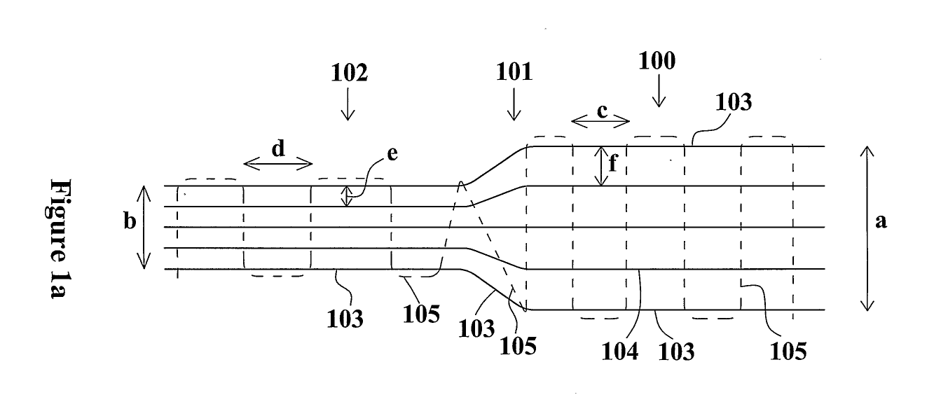

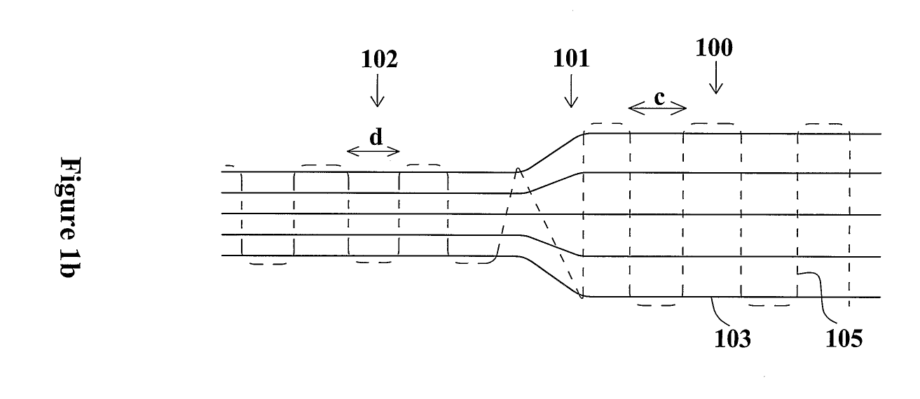

FIG. 1a is a schematic illustration of the surgical cord according to one specific implementation;

FIG. 1b is a schematic illustration of a further specific implementation of the medical cord;

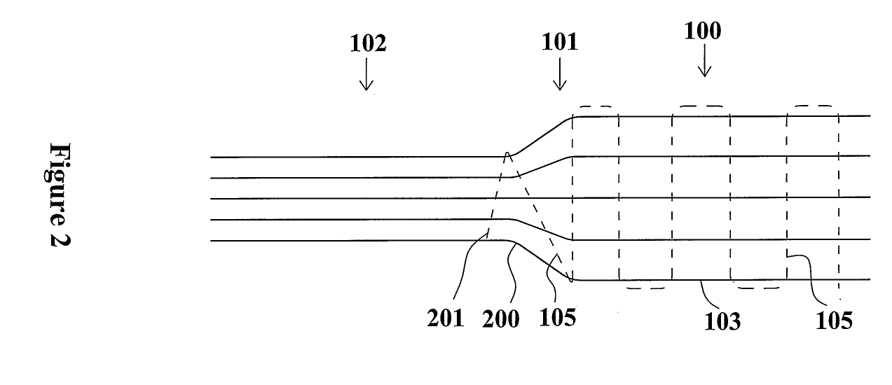

FIG. 2 is a schematic illustration of a further specific implementation of the medical cord;

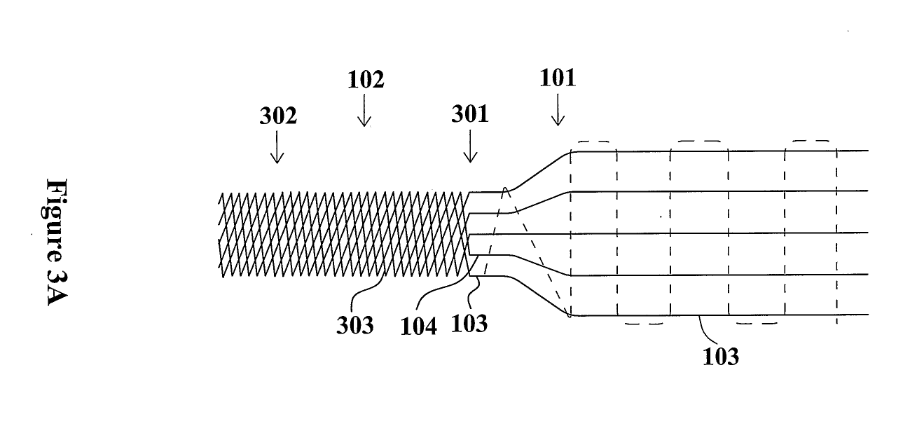

FIG. 3a is a schematic illustration of a further specific implementation of the medical cord;

FIG. 3b is a schematic illustration of a further specific implementation of the medical cord;

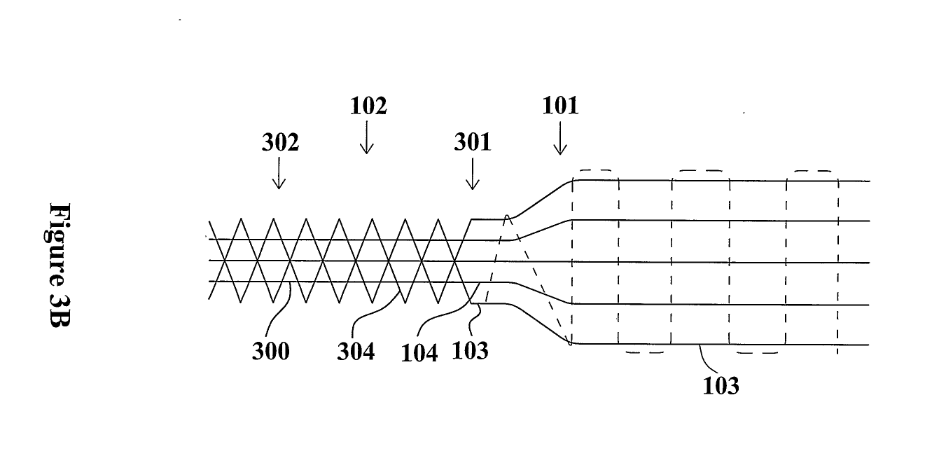

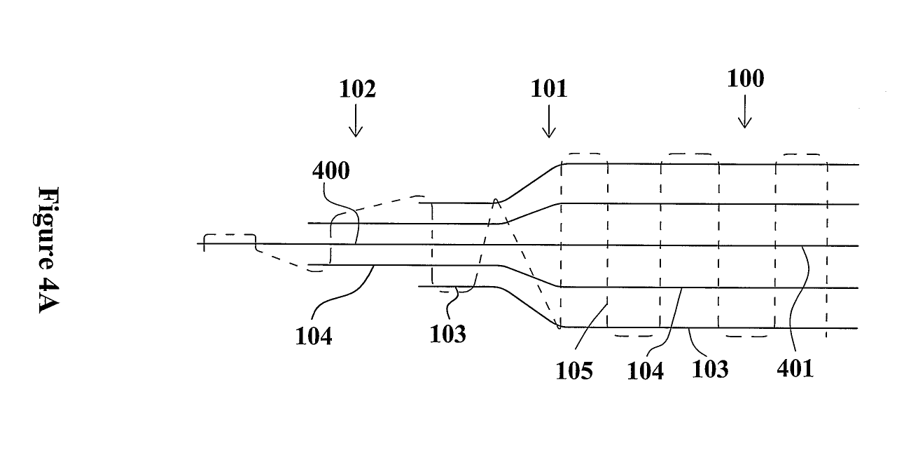

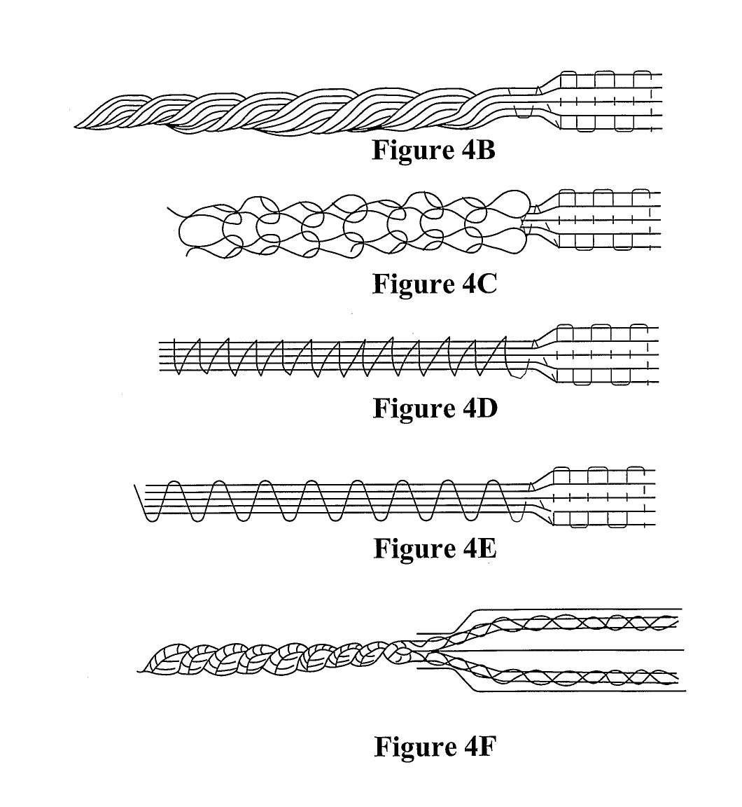

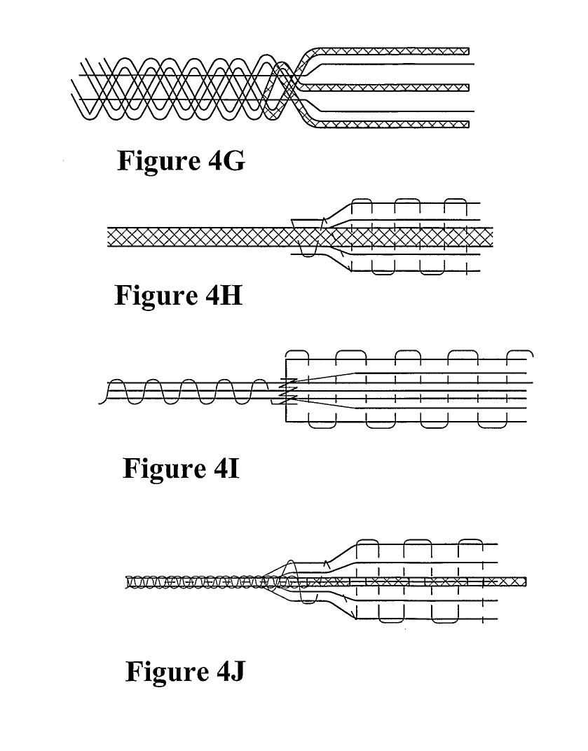

FIG. 4a is a schematic illustration of a further specific implementation of the medical cord;

FIG. 4b is a schematic illustration of a further specific implementation of the medical cord;

FIG. 4c is a schematic illustration of a further specific implementation of the medical cord;

FIG. 4d is a schematic illustration of a further specific implementation of the medical cord;

FIG. 4e is a schematic illustration of a further specific implementation of the medical cord;

FIG. 4f is a schematic illustration of a further specific implementation of the medical cord;

FIG. 4g is a schematic illustration of a further specific implementation of the medical cord;

FIG. 4h is a schematic illustration of a further specific implementation of the medical cord;

FIG. 4i is a schematic illustration of a further specific implementation of the medical cord;

FIG. 4j is a schematic illustration of a further specific implementation of the medical cord;

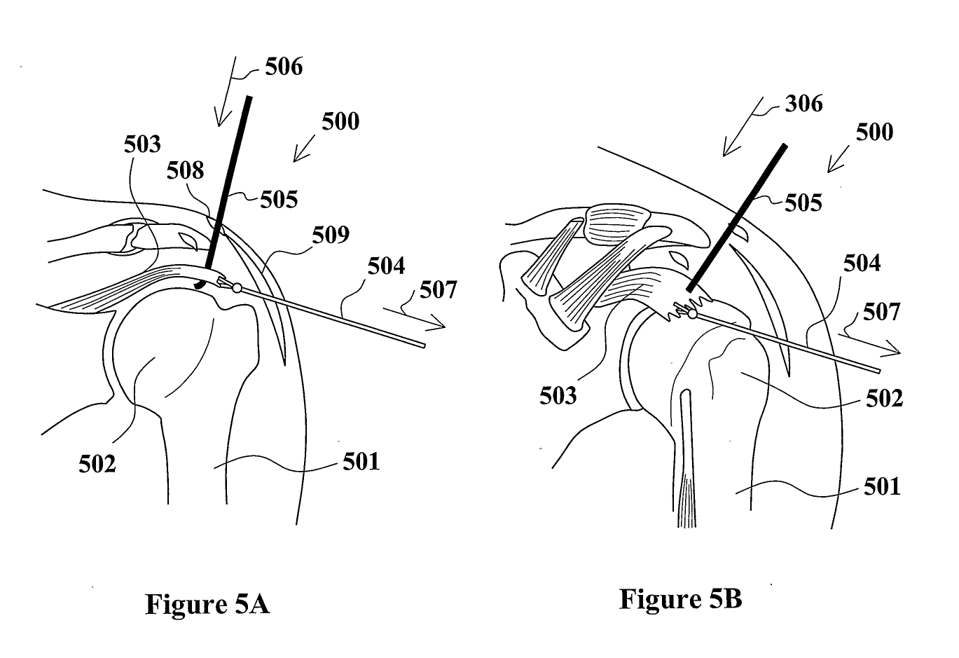

FIG. 5a is a cross sectional side elevation view through the shoulder joint of a human having torn rotary cuff tissue;

FIG. 5b is a perspective view of the shoulder joint of FIG. 5a;

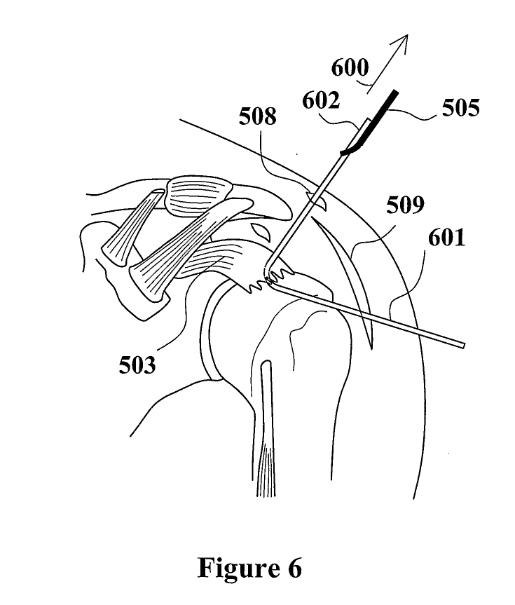

FIG. 6 illustrates the shoulder joint of FIG. 5b during a second stage of the surgical procedure;

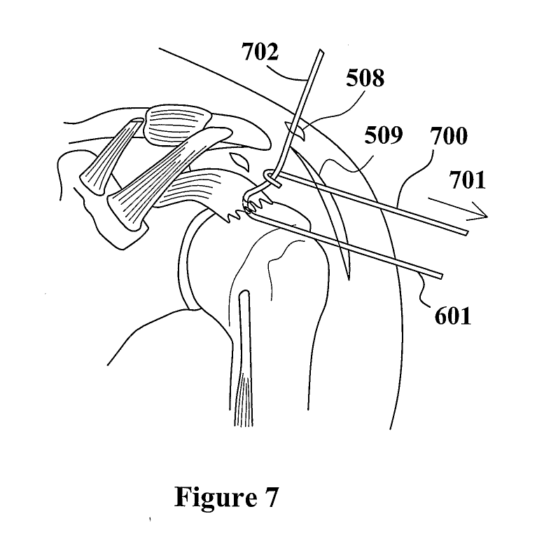

FIG. 7 illustrates the shoulder joint of FIG. 6 during a third stage of the surgical procedure;

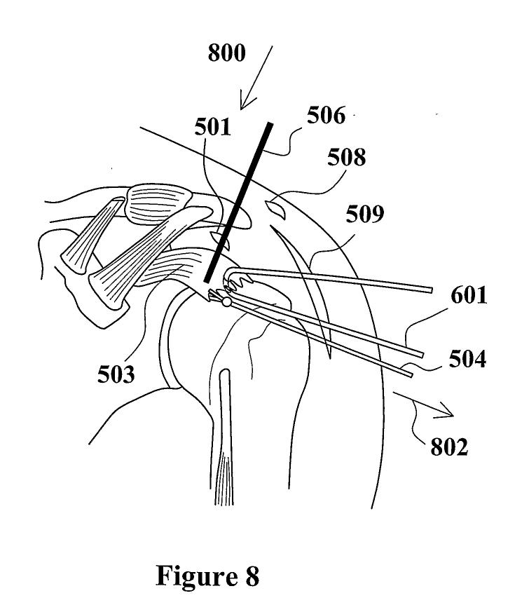

FIG. 8 illustrates the shoulder joint of FIG. 7 during a fourth stage of the surgical procedure;

FIG. 9 illustrates the shoulder joint of FIG. 8 during a fifth stage of the surgical procedure;

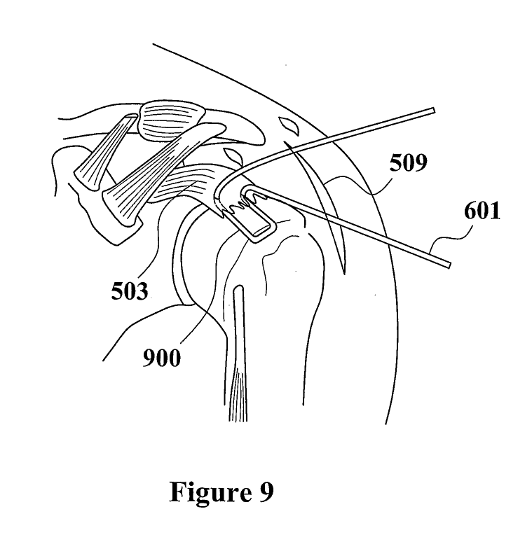

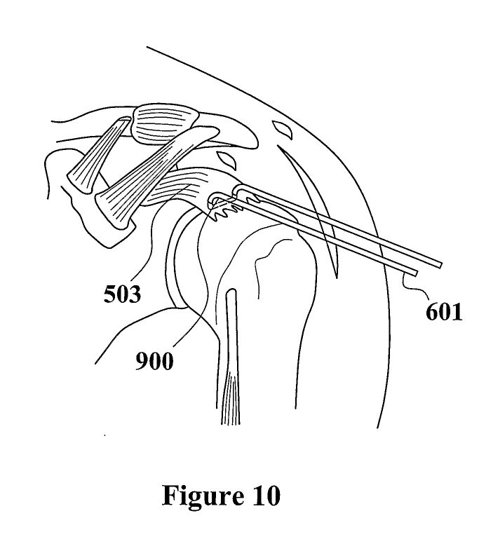

FIG. 10 illustrates the shoulder joint of FIG. 9 during a sixth stage of the surgical procedure;

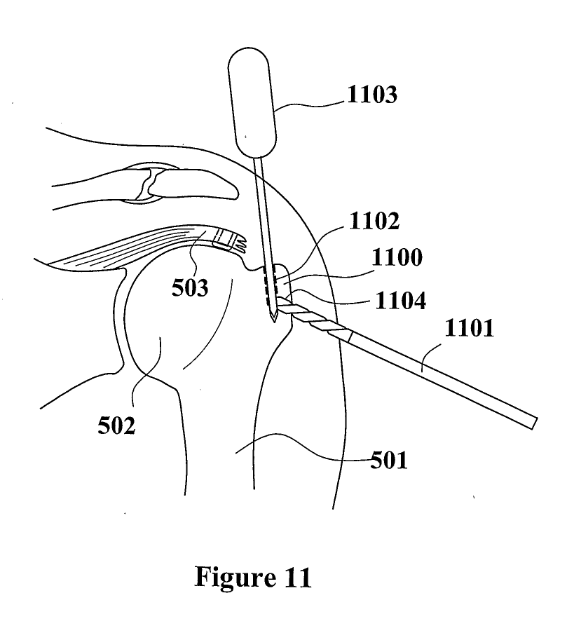

FIG. 11 illustrates a cross section through the shoulder joint according to FIG. 5a during a seventh stage of the surgical procedure;

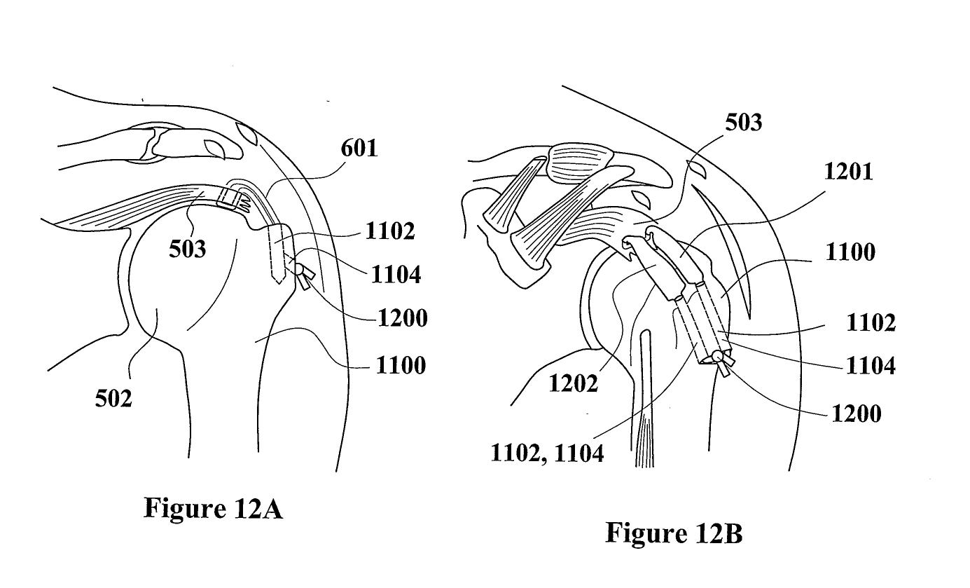

FIG. 12a illustrates the shoulder joint of FIG. 11 at the end of the surgical procedure; and

FIG. 12b illustrates the perspective illustration of the shoulder joint of FIG. 10 at the end of the surgical procedure.

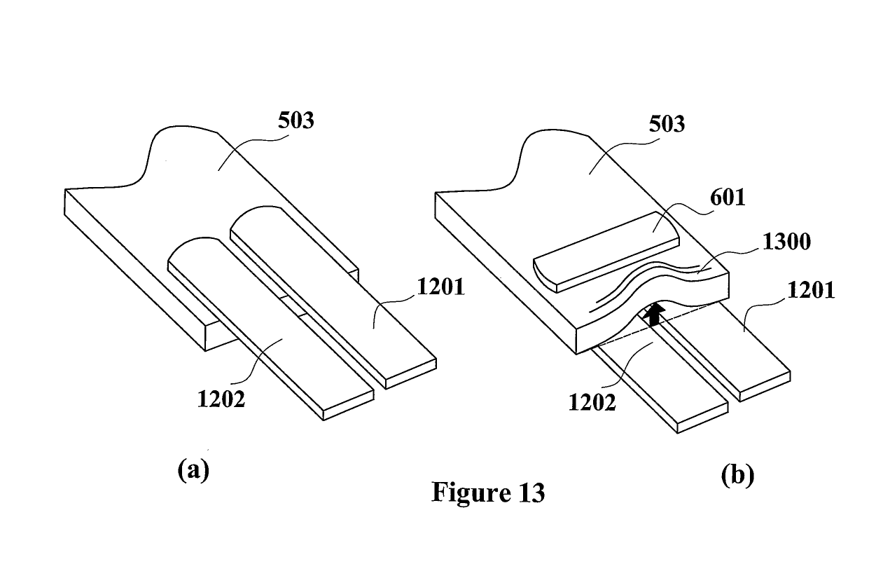

FIG. 13 illustrates alternative positions of the medical cord in the cuff tissue.

Referring to FIG. 1a the surgical cord comprises an interwoven mesh like structure having a plurality of overlapping yarn strands. Each yarn strand comprises a plurality of polyester fibres that are bundled together by twisting or other conventional methods common to the textile industry. The yarn strands are arranged as a plurality of warps 103, 104 aligned substantially parallel with the longitudinal axis of the cord. The strands are also arranged as wefts 105 that overlap and intersect each warp at an angle of 90°. The warps and wefts may be formed by a single piece of yarn or separate yarn pieces, interwoven to create yarn strands, according to conventional textile weaving processes.

The cord comprises outermost warps 103 forming edge regions of the cord and innermost warps 104 forming the inner body of the cord aligned parallel with the outermost warps 103.

The cord comprises a main length 100 and two end regions 102 separated from the main length by tapered regions 101. The main length 100 comprises a width (a) which is greater than a width (b) of end regions 102. The cord width decreases from width (a) to width (b) within tapered region 101.

Warps 103, 104 are separated by distance (f) aligned perpendicular to the main length of the cord within main length region 100. Within this main length region 100 warps 103 are separated by distance (c) aligned with the main axis of the cord. Within end region 102, distance (e) between warps 103, 104 is less than distance (f). The distance (d) between adjacent runs of weft 103 within end regions 102 is greater than distance (c) within the main length according to the specific implementation of FIG. 1a.

FIGS. 1a to 4j are schematic illustrations of the medical cord. According to the preferred embodiment, each weft and warp illustrated within FIGS. 1 to 4j comprise three individual yarn strands positioned side by side. Such that at any one overlapping junction between warp 103, 104 and weft 105, both outermost strands of the set of three warps pass above or below each corresponding outermost strands of the weft whilst respective innermost strands of each warp and weft triad overlap in the opposite direction.

FIG. 1b illustrates a further specific implementation of the medical cord in which the distance (c) between each run of weft 105 within main length 100 is equal to the distance (d) between weft runs within end regions 102.

According to a third embodiment, the weft 105 extends over main length 100 and tapered region 101 and past the interface 200 between the tapered region 101 and end region 102. Weft 105 terminates 201 at end region 102 in close proximity to tapered region 101 such that the majority of the length of each end region 102 is devoid of weft 105. FIG. 3a illustrates the medical cord of FIG. 2 in which the warps 103, 104 within end region 102 are overlapped 303 to form braided section 302. Braiding 303 prevents yarn strands 103, 104 from separating or splitting during manipulation. According to the embodiment of FIG. 3a, a region 301 immediately proximate to tapered region 101 is devoid of braiding 303. In an alternative embodiment the braided region 302 may extend the entire length of each end region 102 such that there is no area devoid of braiding 301. In an alternative embodiment the weft 105 is incorporated into the braided region 302.

FIG. 3b illustrates a variation of the braided end region embodiment of FIG. 3a. According to this embodiment, only the outermost warps 303 are braided together 304 within braided end region 302. Innermost warps 104 are unbraided and extend substantially parallel with the longitudinal axis of the cord. This configuration provides for a thinner end region than the embodiment of FIG. 3a whilst maintaining structural integrity of the yarn strands within end regions 102.

FIG. 4a illustrates a sixth embodiment of the present invention in which the respective length of each warp 103, 104, 401 within the end region 102 differs. This configuration provides a fully tapered width of cord from main length 100 through tapered region 101 and into end regions 102. The amount of cord material therefore decreases within end regions 102 in a direction away from main length 100. Weft 105 is interwoven amongst each warp strand within end region 102 to avoid warps 103 of shorter length from splitting from the main body of cord. The length of the outermost wefts 103 within end region 102 is less than intermediate warps 104 which in turn have a length less than innermost warps 400.

FIGS. 4b to 4j illustrate various different additional embodiments of the present invention in which the cord main body comprises a woven mesh like structure and is bordered at each end by end portions comprising a width being less than that of the main cord length.

The present invention provides a method for the surgical repair of anatomical regions and in particular joints involving cooperation between soft tissue and bone matter. A surgical repair procedure is described with reference to FIGS. 5a to 12b by way of example with reference to the human shoulder joint. The procedure involves repair of torn rotator cuff tissue at the region of the humeral head. The surgical cord of FIGS. 1a to 4j is particularly suitable to repair massive, chronic, retracted tears of the rotator cuff tissue that cannot be mobilised back onto the bone attachment site or if the cuff tissue has undergone degeneration in the case of older patients.

FIGS. 5a and 5b illustrate a cross sectional and perspective view respectively, through a human shoulder region 500 in which the rotator cuff tissue 503 has been torn and separated from the humerus 501 and in particular the humeral head 502. The first stage of the surgical procedure of FIG. 5a follows preparatory steps involving dissection of skin and subcutaneous tissue, the application of a haemostasis and appropriate retractors to separate the humerus from the acromion to enable visualisation of cuff tissue 503.

A stab wound is made through a modified Neviaser portal 508. The cuff tissue 503 is grasped by forceps 504 and pulled 507 to keep it under tension. An artery forceps 505 is passed through the skin, subcutaneous tissue and through the posterior portion of the retracted rotator cuff 503 medial to the tear. The artery forceps 505 is passed through the cuff 503 until the tip of the forceps is visualized through the wound 509.

Referring to FIG. 6, medical cord 601 is introduced into wound 509 and the end of cord 602 is grasped by the artery forceps 505. The artery forceps and the cord end 602 are pulled back through cuff 503 and out of the modified Neviaser portal 508.

Referring to FIG. 7, a hook 700 is brought into position behind cord 601 by insertion through wound 509 to bring the superior arm of the ligament into the wound by drawing hook 700 away from the wound 701 to provide the looped arrangement illustrated in FIG. 8. A stab wound 801 is made through the antero superior portal and the cuff engaged with a grasper or Kocher forceps 504, and pulled 802 to maintain tension in cuff tissue 503. The artery forceps 506 is passed through the skin, subcutaneous tissue and through the retracted anterior portion of the cuff 503, medial to the tear and through the healthy cuff tissue. Forceps 506 are passed through the cuff until the tip of the forceps is visualized through wound 509.

Referring to FIG. 9, the inferior arm cord 601 is passed into the wound and the end of the tape grasped with the artery forceps 506. The artery forceps together with cord 601 are pulled back through the cuff and out of the antra superior portal 801. Accordingly, cord 601 forms a loop 900 at cuff tissue 503, both strands of cord 601 extending from the superior face of cuff tissue 503 as illustrated in FIG. 9. Securing the cord to the cuff tissue by threading the cord through two holes in the cuff tissue to create an n-shaped loop, with two strands of cord extending away from the cuff tissue provides a secure connection between the prosthetic and soft tissue. The use of additional sutures to secure the prosthetic to the tissue is negated providing strength advantages. When each end of cord 601 is pulled to create tension in both strands and cuff material 503, as illustrated in FIG. 10, loop 900 sits against the inferior surface of cuff tissue 503. Cuff 503 is pulled together with prosthetic ligament 601 into the wound using the ends of the ligament 601.

Referring to FIG. 11, a suitable anchor region is identified at the greater tuberosity 110 extending at humeral head 502. In a first stage (not shown) drill 1101 is used to create two parallel aligned boreholes 1102 extending into the greater tuberosity 110. A bone awl 1103 is then inserted into each bore 1102 and an oblique drill hole 1104 is created at the outer side of the humerus to meet the tip of awl 1103. The portal is then marked with a diathermy (not shown). This procedure is used to create two bony tunnels parallel to one another extending through the greater tuberosity.

Referring to FIGS. 12a and 12b, each end of cord 601 is then passed through each respective tunnel 1102, 1104 maintaining the tension in the rotator cuff 503. This can be facilitated by using a suitable suture passer, snare or vinyl suture passer (not shown) through the tunnels. The task of threading cord 601 through tunnels 1102, 1104 is also facilitated by end regions 102 having a width being less than the cord main length 101.

The final stage of the procedure involves pulling cord 601 to ensure cuff tissue 503 has partially covered humeral head 502 and in particular the upper convexity of head portion 502. The two ends of cord 601, emergent from both tunnels 1104 are then tied together in a knot 1200 on the outside of the humerus preferably using a triple knot whilst maintaining the tension in rotator cuff tissue 503. The procedure is completed with a capsular repair over the knot and the layer wise closure of the wound.

Referring to FIG. 12b, the first strand 1201 and second strand 1202 of the present medical cord are aligned side-by-side and substantially parallel between soft cuff tissue 513 and the bone anchor site—corresponding to the bone tunnels 1102, 1104 formed within the greater tuberosity 1100. The combined thickness of strands 1201, 1202 of cord 601 is approximately equal to the width of the cuff tissue 503. The interwoven mesh like structure of cord 601 provides a scaffold for the ingrowth of tissue at the repair site and in particular the interface between prosthetic ligament 601 and cuff tissue 503. The combined thickness of strands 1201, 1202 simulates the cuff tissue 503 to restrict the free movement of the humerus 501 and prevent it from over rotation and subsequent dislocation of the joint.

The tapered end portions of cord 601 allows for the creation of a small knot 1200 which is advantageous to secure the cord in position at humerus 501. The use of prosthetic 601 avoids the risks and problems associated with allografts and avoids donor site morbidity as encountered with autografts.

Preferably, the cord 601 comprises flat tape 10 mm wide by 500 mm long and comprises an open-weave polyester fabric. The drill holes are approximately 3.2 mm in diameter although variation of these dimensions is within the scope of the present invention.

Referring to FIG. 13, the flat tape 601 preferably emerges from the cuff tissue 503 on the superior surface (a). This prevents the lip of the cuff tissue 1300 bulging superiorly which would occur if the flat tape 601 emerged from the inferior surface (b).

1. A surgical cord comprising:
a plurality of interwoven yarn strands comprising warps and wefts extending over a woven main length of the cord, the main length having a width;
two end regions formed by said yarn strands, a width of said end regions being less than the width of the main length wherein said warps extend from said main length in to each end region; and
wherein the main length of the cord comprises a flat, substantially planar profile.

2. The cord as claimed in claim 1 wherein said yarn strands comprise polyester.

3. (canceled)

4. The cord as claimed in claim 1 wherein all of the warps in the main length extend in to each region.

5. The cord as claimed in claim 1 wherein said warps extend over the entire length of each end region.

6. The cord as claimed in claim 1 wherein said wefts extend from said main length in to each end region.

7. The cord as claimed in claim 1 wherein at least a portion of each said end region is braided.

8. (canceled)

9. The cord as claimed in claim 1 wherein the number of warps in each end region is equal to the number of warps in said main length.

10. The cord as claimed in claim 1 wherein a spacing between said yarn strands in each end region is less than the spacing between said yarn strands in said main length.

11. The cord as claimed in claim 1 wherein said wefts do not extend from said main length to each end region.

12. (canceled)

13. (canceled)

14. (canceled)

15. The cord as claimed in claim 1 wherein the yarn strands over at least a portion of each said end region are twisted or knitted.

16. The cord as claimed in claim 1 wherein the yarn strands over at least a portion of each said end region are wrapped.

17. The cord as claimed in claim 1 at least a portion of each said end region comprises a material that is wrapped about said yarn strands.

18. (canceled)

19. The cord as claimed in claim 1 wherein said warps extend over the entire main length and over the entire length of each said end region.

20. The cord as claimed in claim 1 wherein said cord is flat, substantially planar over each said end region.

21. The cord as claimed in claim 1 wherein each end region comprises a substantially uniform width over its respective length.

22. (canceled)

23. A method of surgical repair comprising:
securing a cord to a first biological tissue site, said cord comprising:
a plurality of interwoven yarn strands comprising warps and wefts extending over a woven main length of the cord, the main length having a width;
two end regions formed by said yarn strands, a width of said end regions being less than the width of the main length wherein said warps extend from said main length in to each end region; and wherein the main length of the cord comprises a flat, substantially planar profile;
securing said cord at a second biological tissue site;
wherein said cord extends between said first and second tissue sites.

24. The method as claimed in claim 23 wherein said first tissue site comprises flexible tissue.

25. The method as claimed in claim 23 wherein said second tissue site comprises bone.

26. The method as claimed in claim 23 further comprising:
forming at least one hole at said first tissue site; and
threading said cord through said at least one hole to form a loop in said cord.

27. The method as claimed in claim 23 further comprising:
drilling at least one through bore at said second tissue site;
threading said cord through said at least one through bore; and
securing said cord in position within said at least one through bore to prevent said cord from being retracted from said through bore.

28. A method of connecting a first biological tissue site to a second biological tissue site comprising:
securing a cord to a first tissue site by threading said cord through at least one hole formed in said first tissue site, said cord comprising a plurality of interwoven yarn strands comprising warps and wefts extending over a woven main length of the cord, the main length having a width; two end regions formed by said yarn strands, a width of said end regions being less than the width of the main length wherein said warps extend from said main length in to each end region; and wherein the main length of the cord comprises a flat, substantially planar profile;
securing said cord to a second tissue site;
wherein said cord extends between said first and second tissue sites.

29. The method as claimed in claim 28 wherein said first tissue site comprises rotator cuff tissue.

30. The method as claimed in claim 28 wherein said second tissue site comprises bone.

31. The method as claimed in claim 28 wherein said second tissue site comprises a humeral head wherein said step of securing said cord to said humeral head comprises drilling two through bores through a region of said humeral head and threading end regions of said cord through said through bores and knotting said cord ends together once threaded through said through bores.

32. The method as claimed in claim 28 comprising forming at least two holes in said first tissue and looping said cord through said holes such that said cord extends from said tissue as two strands positioned side by side.

33. The method as claimed in claim 32 wherein the combined width of said two strands is substantially equal to a width of said first tissue.

34. The method as claimed in claim 29 comprising securing said cord to said cuff tissue by passing said cord through an inferior surface of said tissue then a superior surface of said tissue.

35. The method as claimed in claim 29 comprising securing said cord to said cuff tissue by passing said cord through a superior surface of said tissue then an inferior surface of said tissue.
